(19) United States
(12) Patent Application Publication
(10) Pub. No.: US 2021/0069413 A1
(43) Pub. Date: Mar. 11, 2021

(54) Infusion Set with Full Insertion and Removal Action

(71) Applicants: Nicholas John Foley, Edinburgh (GB); Leif Bowman, San Diego, CA (US); Mark H Scott, San Diego, CA (US)

(72) Inventors: Nicholas John Foley, Edinburgh (GB); Leif Bowman, San Diego, CA (US); Mark H Scott, San Diego, CA (US)

(21) Appl. No.: 16/853,247

(22) Filed: Apr. 20, 2020

Related U.S. Application Data
(60) Provisional application No. 62/836,636, filed on Apr. 20, 2019.

(51) Int. Cl. A61M 5/158; A61M 25/06

(52) U.S. Cl. CPC A61M 5/158; A61M 25/0606; A61M 2005/1585; A61M 5/46; A61M 2005/1583; A61M 2230/201; A61M 2005/1586

(57) ABSTRACT

An infusion set has an insertion device that inserts a cannula and introducer needle fully into a patient, and then fully removes the introducer needle with one user motion. In some examples the insertion device does not permit partial insertion or incomplete introducer element removal.

### RELATED APPLICATIONS

This application claims priority to U.S. provisional application No. 62/836,636, filed Apr. 20, 2019, and entitled “An Infusion Set with Full Removal Action,” which is incorporated herein in its entirety. This application is related to U.S. patent application Ser. No. 16/714,701, filed Dec. 14, 2019 and entitled “Kinkless Infusion Set for Medical use,” and to U.S. patent application Ser. No. 16/560,315, filed Sep. 4, 2019 and entitled “Extended Use Infusion Set for Medical Use,” both of which are incorporated herein as if set forth in their entirety.

### FIELD OF THE INVENTION

This invention relates to the needle, cannula, or cannula and introducer needle portion of an infusion set, and in particular insertion devices for infusion sets and sensors, as they relate to medicine delivery, biological fluid gathering, and biological sensing.

### BACKGROUND OF THE INVENTION

A large number of persons in the US and around the world suffer from diseases like diabetes. Such persons achieve treatment through fluid medicines delivered either with injections or through infusion sets.

Infusion sets typically have a duration of 1-3 days before it is necessary to remove the infusion set and find a new location on the body for another infusion set. The result is patients needing between 100+ to 300+ disposable infusion sets annually.

Patients who use do not use infusion sets typically obtain their insulin through multiple daily injections (MDI). These patients have even more frequent and possibly painful injections than those utilizing infusion sets with insulin pumps.

Patients using continuous glucose monitoring (CGM) to measure their glucose levels will change their sensors typically every 3-6 days, although some may not need to change for 12 days. The result is an additional approximately 30-120 injections annually for CGMs with an insertion that is similar to an injection.

The current state of the art for infusion sets uses an introducer needle with a soft cannula around it for the length of the needle that penetrates the skin. The end of the soft cannula is tipped/formed onto the surface of the introducer needle to ensure a tight fit that can withstand the forces of insertion through the skin.

As the introducer needle has a beveled edge to create a sharp point/edge to penetrate the skin, it is necessary for the introducer needle to protrude approximately 3 mm beyond the end of the soft cannula. The result is that a patient who requires a soft cannula tip to deliver medication into the body at a depth of 6 mm must insert 9 mm deep. This creates a deeper wound than necessary and is additionally intimidating to the user.

Further, the introducer needles used in the current infusion sets are seen by the user upon removal, and this view of the needle is intimidating to the user and reinforces the negative aspect of inserting infusion sets.

### SUMMARY OF THE DISCLOSURE

It is an object of the present invention to provide an improved infusion set and/or sensor set which reduces or removes the issues mentioned.

Embodiments of the present invention are directed to an infusion set with the capability to have a full insertion with one user motion.

Embodiments of the present invention are directed to an infusion set with the capability to have a full insertion of a cannula and removal of introducer elements, such as an introducer needle, accomplished with one user motion.

Embodiments of the present invention are directed to an infusion set with the capability to have an insertion device which is manually actuated, and which does not permit a partial insertion.

Embodiments of the present invention are directed to an infusion set with the capability to have an insertion device with a manual actuation motion which transitions from a harder pushing sensation portion to a softer pushing sensation portion.

Embodiments of the present invention are directed to an infusion set with the capability to have a tactile sensation felt by the user when the manual actuation motion of the device which transitions from the harder pushing sensation portion to the softer pushing sensation portion of travel.

Embodiments of the present invention are directed to an infusion set with the capability to have a slow insertion.

A preferred embodiment is directed to an infusion set with the capability to have a controlled slow insertion.

A preferred embodiment is directed to an infusion set with the capability to have a controlled slow insertion in which the timing and speed may be varied by the user.

A preferred embodiment is directed to an infusion set with the capability to have a controlled slow insertion in which actuation speed may be varied by the user, but the insertion timing is not known precisely by the user.

A preferred embodiment is directed to an infusion set with the capability to have a manual actuation of a controlled slow insertion to an effective depth and position of a sensor such as for a continuous glucose monitor (CGM) for the purposes of interaction with biological processes to provide useful data.

### BRIEF DESCRIPTION OF THE DRAWINGS

FIG. 1 is an illustration showing an exemplary embodiment of the present invention with a user-controlled slow insertion.

FIG. 2 is an illustration showing an exemplary embodiment of the present invention with a user-controlled variably slow insertion.

FIG. 3 illustrates an infusion set with a biological sensor, like might be found in a continuous glucose monitor with a user-controlled insertion.

FIG. 4 is an illustration showing an exemplary embodiment of the present invention with a user-controlled slow insertion to a therapeutic depth variably set by the user.

FIG. 5A is an illustration showing an exemplary embodiment with user-controlled manual insertion with one motion and multiple ports in the infusion set for different insertion points within the same site with a biological sensor and a straight, curved, or helical kinkless needle being removed from one insertion port and a different kinkless cannula being inserted, straight or angled, at another insertion port.

FIG. 5B is an illustration showing an exemplary embodiment with user-controlled manual insertion with one motion and multiple ports in the infusion set for different insertion points within the same site with a biological sensor and a straight, curved, or helical kinkless cannula being removed from one insertion port and a different kinkless cannula being inserted, straight or angled, at another insertion port.

FIG. 5C is an illustration showing an exemplary embodiment with user-controlled manual insertion with one motion and multiple ports in the infusion set for different insertion points within the same site with a biological sensor and a straight, curved, or helical kinkless cannula with inserter needle being removed from one insertion port and a different kinkless cannula with inserter needle being inserted, straight or angled, at another insertion port.

FIG. 6 is an illustration showing an exemplary embodiment with user-controlled manual insertion with one motion and multiple ports in the infusion set for different insertion points within the same site with a sensor inserted in one insertion point and a straight, curved, or helical kinkless cannula inserted, straight or angled, at another insertion port.

FIG. 7 is an illustration showing an exemplary embodiment with multiple ports in the infusion set to permit relocation of the sensor and/or straight, curved, or helical needle in the same infusion set at the same site, in order to achieve new depots through new insertions.

FIG. 8A is an illustration showing an exemplary embodiment of the present invention prior to actuation of the inserter.

FIG. 8B is an illustration showing an exemplary embodiment of the present invention during actuation of the inserter.

FIG. 8C is an illustration showing an exemplary embodiment of the present invention after actuation.

FIG. 9A is an illustration showing an exemplary embodiment prior to actuation for a design in which button 985 is fully actuated in its fully pressed position.

FIG. 9B is an illustration showing exemplary embodiment after full actuation for a design in which button 985 is fully actuated in its fully pressed position.

FIG. 9C is an illustration showing exemplary embodiment after full actuation for a design in which button 985 is fully actuated in its fully pressed position. Introducer elements have been fully inserted during travel of the actuation, and the latter portion of the travel of the button causes removal of all introducer elements, and renders the inserter ready for lifting from the body.

FIG. 10A is an illustration showing an exemplary embodiment of the infusion set septum prior to use of the inserter to insert the introducer needle, cannula, and connect the parts of the infusion set with the tubing from the infusion pump.

FIG. 10B is an illustration showing an exemplary embodiment of the infusion set septum after use of the inserter to insert the cannula and insert and remove the introducer needle and connect the parts of the infusion set with the tubing from the infusion pump.

FIG. 11A has illustration views showing an exemplary embodiment of the extended use infusion set with variable user-controlled manual insertion in accordance with the present invention.

FIG. 11B is an illustration showing an exemplary embodiment of the extended use infusion set with curved or helical cannula inserted at therapeutic depth.

FIG. 11C is an illustration showing an exemplary embodiment of the extended use infusion set with curved cannula inserted at therapeutic depth and the inserter removing the insertion needle and any other introducer elements as the last part of its one-motion full insertion and removal.

### DETAILED DESCRIPTION OF THE EXEMPLARY EMBODIMENTS

The embodiments and examples shown here are to provide enough information to fully understand the invention. One skilled in the art will understand how minor changes or deviations can be made and still be within the scope of the invention. The following description of exemplary embodiments of the invention is not intended to limit the scope of the invention to these exemplary embodiments, but rather to enable any person skilled in the art to make and use the invention. To assist in a clear and unambiguous understanding of the disclosure, the following definitions are used:

Definitions
A bolus is generally a single dose of insulin, typically a larger dose administered at once and often at mealtime. The purpose is to provide additional insulin to help the body address the carbohydrate intake of the meal. A bolus may also be given if there are other foreseen or unforeseen events affecting blood sugar. A bolus may be administered via an infusion set which also delivers basal insulin, or may be injected separately.
The basal insulin level is the amount of insulin referred to as necessary to address the diabetic patient's need for insulin between meals or other foreseen or unforeseen events, such as mealtime or snacks. The basal insulin dosage is often a lower level, delivered more regularly or nearly continuously, between meals.
A cannula is similar to a hollow metal needle, but often made of a softer, flexible material which can bend. Some cannula designs incorporate an introducer needle, which may or may not be hollow, to help the cannula achieve its desired position.
An introducer needle is a hollow or solid needle which generally does not deliver medicine itself, but instead helps the cannula penetrate the skin and achieve its desired position to deliver medicine for therapeutic benefit at a subdermal or subcutaneous depth and position.
A needle is a sharp object which is used to penetrate the skin and push through the body to a desired depth or position. A needle may be hollow or solid. It may allow medicine to travel through it into the body, or it may act only as an introducer needle for a softer and/or more flexible cannula and be retracted after its positioning function, or it may perform both medicine delivery and introducer functions.
A depot is a location where medicine is delivered by the needle or cannula, and generally the “depth and location” within the patient tissue where the medicine is absorbed by the body.
An insertion device is a part of an infusion set which may be used to assist the attachment of the affixed portion of the infusion set, and the insertion of the cannula and inserter needle, and may be discarded or reused after actuation. Methods and mechanisms described herein to insert the cannula and fully remove introducer elements entirely may be in the affixed portion of the infusion set, or may be resident in the insertion device, or some combination.
An introducer element is a part of the infusion set or its insertion device which is instrumental in placing the needle, cannula, or other part that remains within the body after insertion into proper position. An insertion element does not itself remain within the body after insertion, and will instead be removed after positioning, as the insertion device completes its function.

As shown in the included figures, the illustrations depict instances of infusion sets inserted into the skin for the purposes of delivering fluid medicine into the tissue beneath the outer layer of skin, such as insulin for the treatment of diabetes subcutaneously. However it will be understood that the invention may also be utilized for delivery of other medicine, hormones, vitamins, saline, including fluids containing dissolved or suspended solids if in the future such a treatment is created. The invention may be used for the placement of sensors capable of measuring biological information, such as glucose levels, salinity, red or white blood cells, T-cell counts, dissolved oxygen, ketones, lactate, or the like on a continuous or intermittent basis, whether for information, entertainment, or compliance purposes only, as part of a feedback loop in medicine delivery, or to aid in a combination of manual and automated administration of fluid described above, whether that manual administration is through the infusion set or administered elsewhere on the body.

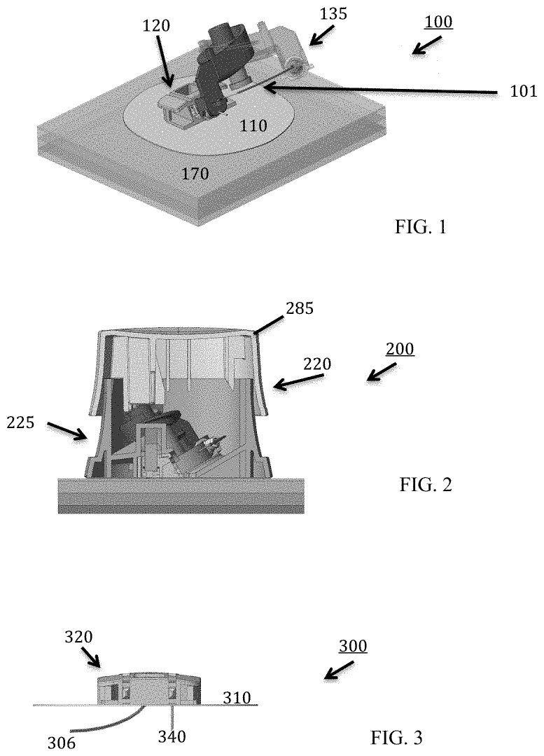

FIG. 1 shows an infusion set 100 with a curved cannula 101, which may be slowly inserted by the user to a depth at which one or more therapeutic doses of medicine may be delivered. The adhesive pad is 110, the head is 120, and the inserter mechanism is 135. The skin is 170, The skin layers are (top to bottom) epidermis, dermis, subcutaneous tissue, muscle.

FIG. 2 shows an infusion set 200 with a curved kinkless cannula, which may be slowly inserted with variable speed by the user to a depth at which one or therapeutic doses of medicine may be delivered. The head is 220. Button 285 is in its initial position, prior to actuation of the inserter. A user depresses the button to insert the cannula under their skin. The base is part 225. The skin layers are (top to bottom) epidermis, dermis, subcutaneous tissue, muscle.

FIG. 3 shows an infusion set 300 with a biological sensor 340, which may be slowly inserted to an effective depth for sensing biological activity or levels that may be utilized for entertainment or information purposes only, or may also be used for the wearer or others to inform or control manually administer medicine at the site or elsewhere, or be used by devices attached to the same or another infusion set to provide intermittent or continuous fluid delivery. The cannula is 306, the adhesive pad is 310 and the head is 320.

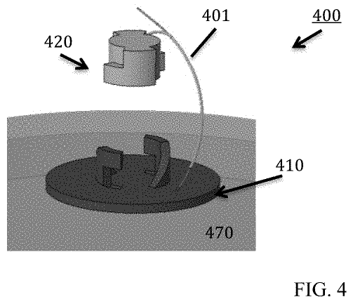

FIG. 4 shows a helical needle 401 on infusion set 400 which may slowly be inserted to a therapeutic depth variably set by the user. The adhesive pad is 410, the head is 420. The skin is 470.

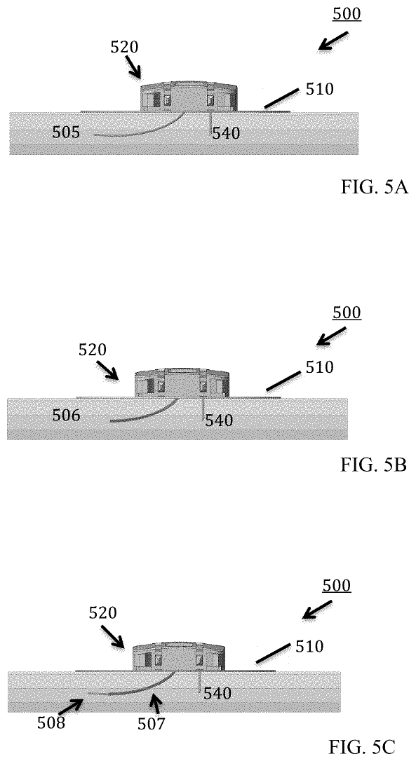

FIG. 5A shows an exemplary embodiment of the infusion set 500 with a kinkless curved needle 505 with a biological sensor 540, like might be found with a continuous glucose monitor. The wearer may prefer having both sensor and medicine delivery on the same infusion set due to reduction in complexity and longer time between site reuse, since only one site is used at a time. The adhesive pad is 510 and the head is 520. The skin layers are (top to bottom) epidermis, dermis, subcutaneous tissue, muscle.

FIG. 5B shows an exemplary embodiment of the infusion set 500 with a kinkless curved cannula 506 with a biological sensor 540, like might be found with a continuous glucose monitor. The wearer may prefer having both sensor and medicine delivery on the same infusion set due to reduction in complexity and longer time between site reuse, since only one site is used at a time. The adhesive pad is 510 and the head is 520. The skin layers are (top to bottom) epidermis, dermis, subcutaneous tissue, muscle.

FIG. 5C shows an exemplary embodiment of the infusion set 500 with a kinkless curved cannula 507 with introducer needle 508 with a biological sensor 540, like might be found with a continuous glucose monitor. The wearer may prefer having both sensor and medicine delivery on the same infusion set due to reduction in complexity and longer time between site reuse, since only one site is used at a time. The adhesive pad is 510 and the head is 520. The skin layers are (top to bottom) epidermis, dermis, subcutaneous tissue, muscle.

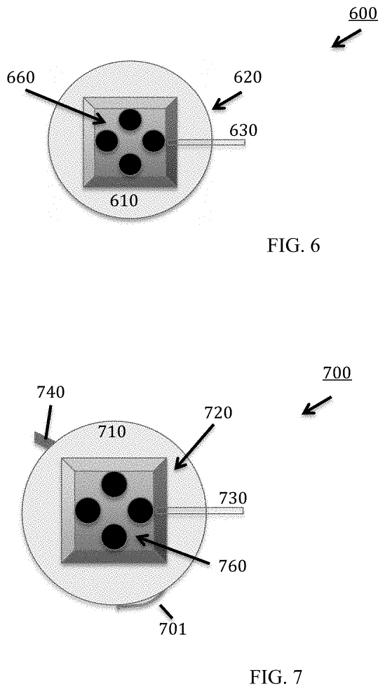

FIG. 6 shows an exemplary embodiment of the infusion set 600 with multiple ports 660 in the infusion set to permit relocation of the straight, curved or helical needle in the same infusion set at the same site, in order to achieve new depots through new insertions. The adhesive pad is 610, the head is 620, and the tubing to the infusion pump is 630.

FIG. 7 shows an exemplary embodiment with a multiple ports 760 in the infusion set 700 to permit relocation of the sensor 740 and/or straight, curved, or helical needle 701 in the same infusion set at the same site, in order to achieve new depots through new insertions. It may be necessary or desired to relocate the sensor, or further insert or partially remove the sensor to sense different biological processes or other reasons. It may be necessary or desired to relocate the needle, cannula, or cannula with inserter needle to another port to achieve a new depot for increased therapeutic delivery over the original depot. The adhesive pad is 710, the head is 720, and the tubing to the infusion pump is 730. Advantageously, the same hole may be reused, in some cases. Advantageously, some constructions of cannula and/or sensor may permit two or more items to be inserted through the same port.

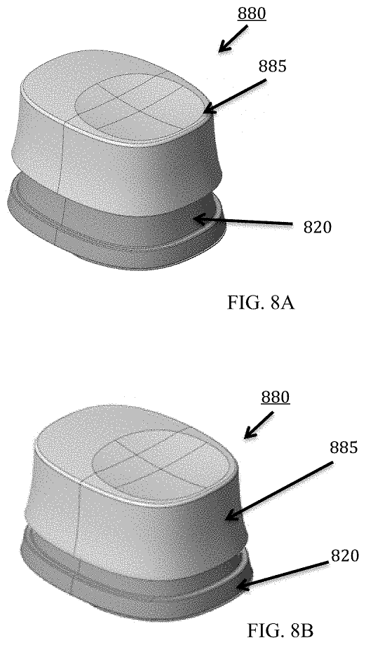

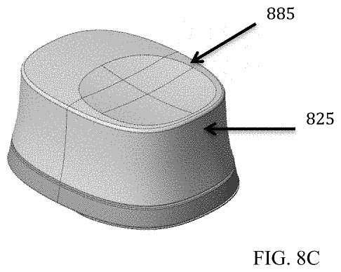

FIG. 8A shows an exemplary embodiment of an insertion device 880 in which button 885 is in its initial position, prior to actuation of the inserter. The base is part 820. Device 880 enables the insertion of the cannula and introducer needle and after they are fully inserted, the removal of the introducer needle, all in one motion. Fore example, a patient may place the infusion set on their abdomen and press the button 885. Pressing the button will first insert the cannula/needle pair, and upon full insertion will act to then remove the introducer needle. Although device 880 is illustrated with a push button, it ill be understood that other activation devices can be used, such as knobs and levers.

FIG. 8B shows an exemplary embodiment 880 of an insertion device in which button 885 is in its partially actuated position, as the cannula and any introducer elements have been fully inserted, and prior to the full activation of button 885, which causes the removal of all introducer elements. The base is part 820. As illustrated, partial activation of the button 885 has fully inserted the cannula/needle pair, and further activation of the button will act to remove the introducer needle. It will be understood that in the alternative pressing the button 885 could be such that the cannula/needle pair is only fully inserted when the button 885 is fully engaged. In this case, an internal spring or other energy-capturing device would capture kinetic energy, and upon full insertion, the captured energy would be released to remove the introducer needle. It will be understood that several alternatives exist to capture energy within the insertion device.

FIG. 8C shows an exemplary embodiment 880 of an insertion device in which button 885 has been released, as the cannula has been left inserted under the skin and any introducer elements have been fully removed from the body. Button 885 is shown in its fully actuated position, as the cannula and any introducer elements have been fully inserted during travel of the actuation, and the latter portion of the travel of the button 885 causes removal of all introducer elements, and renders the inserter ready for lifting from the body. The button 885 is locked down to indicate a used device and prevent secondary needle actuation. The base is part 820.

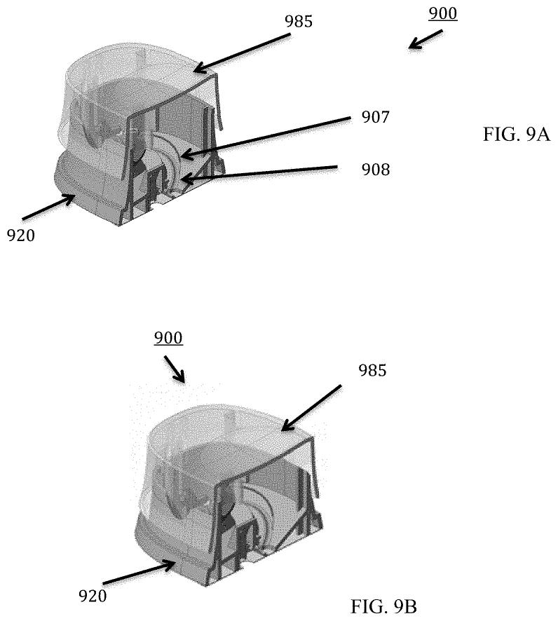

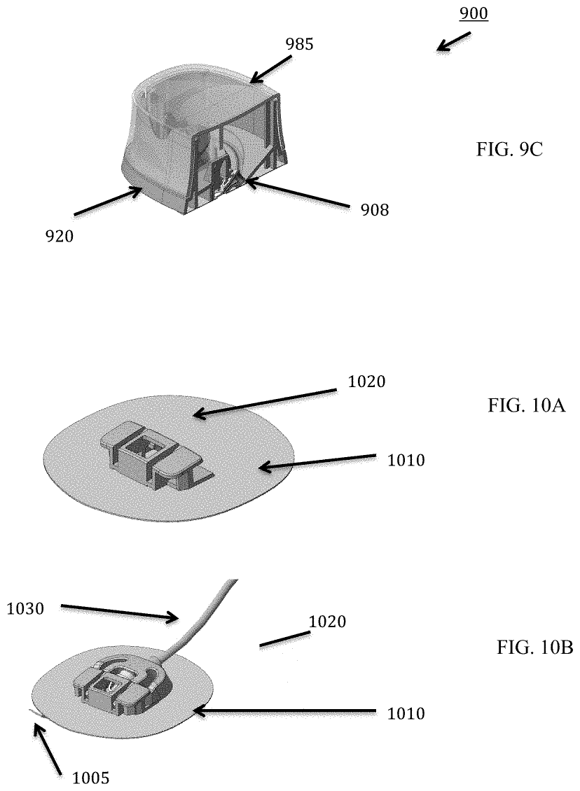

FIG. 9A shows an exemplary embodiment of an insertion device 900 in which button 985 is in its initial position, prior to actuation of the inserter. The cannula 907 and introducer needle 908 are visible in their initial position ready to be inserted. The base is part 920. Device 900 enables the insertion of the cannula and introducer needle and after they are fully inserted, the removal of the introducer needle, all in one motion. For example, a patient may place the infusion set on their abdomen and press the button 985. Pressing the button will first insert the cannula/needle pair, and upon full insertion will act to then remove the introducer needle. Although device 900 is illustrated with a push button, it ill be understood that other activation devices can be used, such as knobs and levers.

FIG. 9B shows an exemplary embodiment 900 of an insertion device in which button 985 is in its partially actuated position, as the cannula and any introducer elements have been fully inserted, and prior to the full activation of button 985, which causes the removal of all introducer elements. The base is part 920. As illustrated, partial activation of the button 985 has fully inserted the cannula/needle pair, and further activation of the button will act to remove the introducer needle. It will be understood that in the alternative pressing the button 985 could be such that the cannula/needle pair is only fully inserted when the button 985 is fully engaged. In this case, an internal spring or other energy-capturing device would capture kinetic energy, and upon full insertion, the captured energy would be released to remove the introducer needle. It will be understood that several alternatives exist to capture energy within the insertion device.

FIG. 9C shows an exemplary embodiment 900 after full actuation for a design in which button 985 is fully actuated in its fully pressed position. Button 985 is shown in its fully actuated position, as the cannula and any introducer elements have been fully inserted during travel of the actuation, and the latter portion of the travel of the button 985 causes removal of all introducer elements, and renders the inserter ready for lifting from the body. The cannula and septum module are fully inserted and the fully removed introducer needle 908 is visible. The button 985 is locked down to indicate a used device and prevent secondary needle actuation. The base is part 920.

FIG. 10A shows an exemplary embodiment of the infusion set hub 1020 prior to use of the inserter to insert the introducer needle, cannula, and connect the parts of the infusion set with the tubing from the infusion pump. The adhesive pad is 1010.

FIG. 10B shows an exemplary embodiment of the infusion set hub 1020 after use of the inserter to insert the cannula and to insert and remove the introducer needle and connect the tubing 1030 to the infusion pump with the tubing from the infusion pump. The adhesive pad is 1010 and the cannula is 1005.

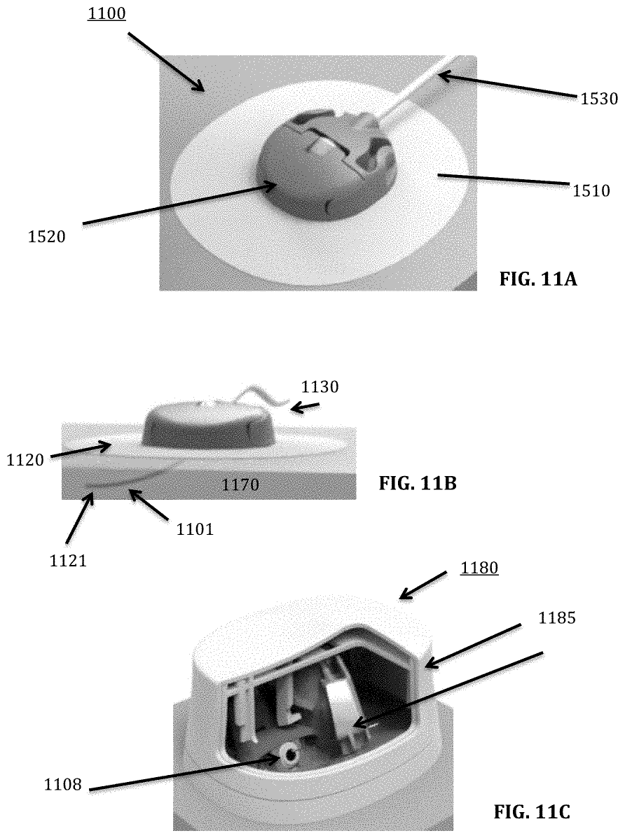

Referring now to FIG. 11A, an infusion set 1100 is illustrated, attached with adhesive pad 1110 and with head piece 1120 connected through tubing 1130 to the source of medication. Infusion set 1100 is designed and intended to insert a cannula subcutaneously into a human at user-controlled variable-speed insertion to enable a medicine or therapeutic to be delivered into the patient at a desirable depth and position. As illustrated, the infusion set 1100 has already been inserted under the patient's skin to an initial depth and position. Example insertion mechanisms are illustrated in following sections.

Referring now to FIG. 11B, the infusion set head piece 1120 is connected through tubing to the source of the medication, which in some cases may be insulin driven from an infusion pump. Head piece 1120 also connects to the cannula 1101. Cannula 1101 may be made from metal, plastics, or other materials appropriate for delivery of medication. It will be understood that the selection of material for cannula 1101 may be particularly selected for the type of medication to be delivered. For example, some chemotherapy medications may degrade certain materials, so materials resistant to chemical damage would need to be selected. It will also be understood that the cannula 1101 may use an introducer needle to initially place the cannula 1101 to its desired position or location 1121 into the skin 1170, with the introducer needle then being removed and discarded.

Referring now to FIG. 11C, an exemplary embodiment of the insertion device 1180 is shown after full actuation for a design in which button 1185 is fully actuated in its fully pressed position. Button 1185 is shown in its fully actuated position, as the cannula and any introducer elements have been fully inserted during user-controlled variable speed travel of the actuation, and the latter portion of the travel of the button 1185 causes removal of all introducer elements, and renders the inserter ready for lifting from the body. The inserter mechanism 1135 has caused the cannula and septum module to be fully inserted and the fully removed introducer needle 1108 is visible. The button 1185 is locked down to indicate a used device and prevent secondary needle actuation.

Common and familiar methods and assemblies may not be mentioned, in order to be brief and clear. While particular preferred and alternative embodiments of the present intention have been disclosed, it will be appreciated that many various modifications and extensions of the above described technology may be implemented using the teaching of this invention. All such modifications and extensions are intended to be included within the true spirit and scope of the appended claims.

What is claimed, is:

1. An infusion set having a full insertion and full removal pathway, comprising:
a base portion having an adhesive backing that adheres to a patient's skin;
a disposable insertion device which may be connected to the base portion during the insertion of the cannula and introducer elements;
an insertion mechanism in the insertion device constructed to insert a cannula/introducer needle pair under the skin of a patient;
a removal mechanism in the insertion device constructed to remove the introducer needle;
an actuating device in the insertion device that is engageable by the patient or caregiver, the actuating device coupled to the insertion mechanism and to the removal mechanism; and
wherein the actuation device is constructed to first engage the insertion mechanism to insert the cannula/needle pair, and later to engage the removal mechanism to remove the introducer needle.

2. The infusion set according to claim 1, wherein the actuating device causes the insertion of the cannula/needle pair to be completed in a single motion by the patient.

3. The infusion set according to claim 1, wherein the actuating device causes both (a) the insertion of the cannula/needle pair to be completed and (b) the removal of the introducer needle to be completed in a single motion by the patient.

4. The infusion set according to claim 3, wherein the removal mechanism is activated responsive to the continued engagement of the actuation device by the patient.

5. The infusion set according to claim 3, wherein during insertion of the cannula/needle pair the removal mechanism stores kinetic energy that the removal mechanism uses to remove the introducer needle.

6. The infusion set according to claim 5, wherein the kinetic energy is stored in a spring in the head portion.

7. The infusion set according to claim 6, wherein the kinetic energy is released upon the insertion mechanism having fully inserted the cannula/needle pair.

8. The infusion set according to claim 1, wherein the activation mechanism locks into position upon the insertion mechanism having fully inserted the cannula/needle pair.

9. The infusion set according to claim 1, wherein the actuating device is a lever, push button or knob.

10. An infusion set having a full insertion and full removal pathway, comprising:
a base portion having an adhesive backing that adheres to a patient's skin;
a disposable insertion device which may be connected to the base portion during the insertion of the cannula and introducer elements;
an actuating device in the insertion device that is engageable by the patient or caregiver;
a cannula and introducer needle pair in the insertion device when the actuating device is in its initial position;
wherein when partially engaged, the actuating device has operably engaged the cannula/needle pair to insert the cannula/needle pair fully under the skin of the patient; and
wherein when fully engaged and in its final position, the actuating device has operably engaged the introducer needle pair to remove the introducer needle from the patient and back into the disposable insertion device.

11. The infusion set according to claim 10, wherein the activation mechanism locks into position upon the insertion mechanism having fully inserted the cannula/needle pair.

12. The infusion set according to claim 10, wherein the actuating device is a lever, push button or knob.

13. The infusion set according to claim 10, wherein the actuating device causes both (a) the insertion of the cannula/needle pair to be completed and (b) the removal of the introducer needle to be completed in a single motion by the patient.

14. An infusion set having a full insertion and full removal pathway, comprising:
a base portion having an adhesive backing that adheres to a patient's skin;
a disposable insertion device which may be connected to the base portion during the insertion of the cannula and introducer elements;
an actuating device in the insertion device that is engageable by the patient or caregiver;
a cannula and introducer needle pair in the insertion device when the actuating device is in its initial position;
an energy capture mechanism that stores kinetic energy as the actuating device is moved from its initial position to its final position;
wherein when fully engaged and in its final position, the actuating device has operably engaged the cannula/needle pair to insert the cannula/needle pair fully under the skin of the patient; and
wherein after the cannula/needle pair is fully inserted, the energy capture mechanism releases energy to remove the introducer needle from the patient and back into the disposable insertion device.

15. The infusion set according to claim 14, wherein the energy capture device is a spring.

16. The infusion set according to claim 14, wherein the activation mechanism locks into position upon the insertion mechanism having fully inserted the cannula/needle pair.

17. The infusion set according to claim 14, wherein the actuating device is a lever, push button or knob.

18. The infusion set according to claim 14, wherein the actuating device causes both (a) the insertion of the cannula/needle pair to be completed and (b) the removal of the introducer needle to be completed in a single motion by the patient.
